SEQUENCE LISTING (6 sequences)

SEQ ID NO: 1 - DNA, 19 bp, Artificial
  TMO1
accatgaacggtaaaggtt

SEQ ID NO: 2 - DNA, 20 bp, Artificial
  TMO2
tccatcttggagataccatt

SEQ ID NO: 3 - DNA, 20 bp, Artificial
  TERT
tccactcgcgrggaccggcc

SEQ ID NO: 4 - DNA, 20 bp, Artificial
  SCR_1
acacggatatcggtaagaat

SEQ ID NO: 5 - DNA, 20 bp, Artificial
  SCR_2
gacttagacaattaacgaag

SEQ ID NO: 6 - DNA, 20 bp, Artificial
  NTC
gagtcattcgacttctgact

(19) United States
(12) Patent Application Publication
(10) Pub. No.: US 2023/0257741 A1
(43) Pub. Date: Aug. 17, 2023

(54) Method for Retaining Splicing RNAs in the Nucleus Based on Chemically Modified Antisense Oligonucleotides

(71) Applicant: The Regents of the University of Colorado, a body corporate, Denver, CO (US)

(72) Inventors: Gabrijela Dumbovic, Boulder, CO (US); John Rinn, Boulder, CO (US); Marvin Caruthers, Boulder, CO (US); Katarzyna Jastrzebska, Brzeziny (PL); Heera K. Langner, Thornton, CO (US)

(21) Appl. No.: 17/759,402

(22) PCT Filed: Jan. 29, 2021
(86) PCT No.: PCT/US21/15673

§ 371 Date: Jul. 25, 2022

Related U.S. Application Data
(60) Provisional application No. 62/967,221, filed on Jan. 29, 2020.

(51) Int. Cl. C12N 15/113; A61K 31/7115; A61P 35/00

(52) U.S. Cl. CPC C12N 15/113; A61K 31/7115; A61P 35/00

(57) ABSTRACT

The inventive technology relates to systems and methods for the use of chemically modified antisense oligonucleotides (ASOs) to sterically prevent intron excision thus causing nuclear retention of the RNA and inhibiting nuclear export. In preferred therapeutic embodiments, ASOs may be engineered to quarantine clinically relevant RNAs in the nucleus of the cell.

### CROSS-REFERENCE TO RELATED APPLICATION

This International PCT Application claims the benefit of and priority to U.S. Provisional Application No. 62/967,221, filed Jan. 29, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 15, 2022, is named “90245-00452-Sequence-Listing” and is 1,392 bytes in size.

### TECHNICAL FIELD

This invention relates generally to the field of ribonucleic acid (RNA) therapeutics, and in particular chemically modified antisense oligonucleotides (ASOs) to sterically prevent intron excision thus altering subcellular localization of pre-processed RNAs.

### BACKGROUND

One of the biggest revelations of the human genome project was that the vast majority of the genome is non-coding, and importantly, that the majority of those non-coding regions are transcribed. One such class of non-coding transcripts is long non-coding RNAs (lncRNAs). Recently, it has been determined that a substantial fraction of mutations underlying human diseases arise from the non-coding genome, in many instances involving lncRNA genes. One such lncRNA is the Taurine Upregulated Gene 1 (TUG1) known for its role in multiple cellular processes, in gene regulation in the nucleus and regulation of male fertility. Identifying lncRNAs as relevant mediators in disease opened the necessity and new avenues for RNA-based therapies. Advantages of RNA-based therapies are substantial, mainly because these approaches do not modify the DNA, while the therapeutic agent is diluted out over time. Thus, an application for transient RNA targeting to restore cell physiology would be a safe and effective route to therapy. The use of antisense oligonucleotides (ASOs) for steric blockage of certain domains within an RNA in order to restore its function has been intensively explored during the last decades. A new generation of ASOs was developed by chemically altering the natural nucleic acid structure to avoid degradation by intracellular nucleases and to increase affinity to RNA. The first use of ASOs as a therapeutic tool for genetic diseases was done in thalassemia where an abnormal splicing event was corrected with ASOs. Since then, a progressive number of studies applied ASOs to modulate splicing in diseases such as the Duschenne Muscular dystrophy, NF 1 and Hutchinson-Gilford syndrome. The location of RNA within the cell is a critical factor determining its corresponding functions. To date, the use of ASOs for blocking splicing, nuclear export and hence altering subcellular localization of RNA has not been explored.

### SUMMARY OF THE INVENTION

One aspect of the inventive technology includes the novel use of chemically modified ASOs, and preferably one or more TMOs that are configured to be complementary to a portion of a target RNA such that the complementary hybridization sterically prevents intron excision and inhibits nuclear export of target RNA.

Another aspect of the inventive technology includes the novel use of chemically modified ASOs, and preferably one or more TMOs that are configured to be complementary to a portion of a target RNA having an intro and exon portion such that the complementary hybridization sterically prevents intron excision and inhibits nuclear export of target RNA.

Another aspect of the inventive technology includes the novel use of chemically modified ASOs, and preferably one or more TMOs that are configured to be complementary to a portion of a target lncRNA having an intro and exon portion such that the complementary hybridization sterically prevents intron excision and inhibits nuclear export of target lncRNA.

Another aspect of the inventive technology includes the novel use of chemically modified ASOs, and preferably one or more TMOs that are configured to be complementary to a portion of a target RNAs associated with a RNA localization disease condition, such as TUG1, TERT, or KRAS having an intro and exon portion such that the complementary hybridization sterically prevents intron excision and inhibits nuclear export of target RNAs into the cytoplasm for translation.

Another aspect of the inventive technology includes the novel therapeutic use of chemically modified ASOs, and preferably one or more TMOs to sequester target RNA that may be clinically significant in RNA localization disorders such as cancer.

Another aspect of the inventive technology includes the novel therapeutic use of chemically modified ASOs, and preferably one or more TMOs to sequester target lncRNA that may be clinically significant in RNA localization disorders such as cancer.

Another aspect of the inventive technology includes the novel therapeutic use of chemically modified ASOs, and preferably one or more TMOs to sequester target TERT, and preferably the nuclear localization of TERT, and further using this aspect as a novel treatment of cancers that implicate TERT dysregulation.

Another aspect of the inventive technology includes the novel use of chemically modified ASOs, and preferably one or more TMOs that are configured to be complementary to a portion of a TERT. In a preferred embodiment, the TMOs hybridize with a TERT exon/intron donor splice site and prevent TERT splicing, and further localize TERT to the nucleus.

Another aspect of the inventive technology includes ASOs SEQ ID NO. 1-3, which may further include one or more thiomorpholino linkages.

This summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this summary as well as in the attached drawings and the description of the various embodiments of the invention and no limitation as to the scope of the present disclosure is intended by either the inclusion or non-inclusion of elements, components, etc. in this Summary.

Additional aspects of the present disclosure will become more readily apparent from the detailed description below, particularly when taken together with the drawings.

### BRIEF DESCRIPTION OF FIGURES

FIG. 1 (A) The chemical structure of thiomorpholino oligonucleotide (TMO). (B) The design of TUG1 TMO1 (SEQ ID NO. 1) and TMO2 (in red) (SEQ ID NO. 2) against the donor splice sites. For TMOs, upper-case red letters refer to thiomorpholino nucleotides and lower-case letters to 2′-deoxynucleosides at the 3′ end of each TMO. (C) Experimental setup to assess the efficiency of TMO-based intron inclusion and its effect of subcellular localization of TUG1 and cell growth. (D) TMO location scheme in respect to TUG1 transcript and the location on intron spanning primers (not to scale). (E) PCR product of the intron spanning RT PCR of untreated (NT), control TMO (Ctrl) and increasing doses of a mixture of TUG1 TMO1 and TMO2. Black arrow, spliced product; red arrow, unspliced product. Below, the percentage of unspliced product. (F) UCSC browser displaying Sanger sequencing results of spliced (band 1) and unspliced (band 2) products for intron 1 RT PCR (on top). Below, the sequences for spliced (band 3.1 and band 3.2) and unspliced (band 4) products for intron 2 RT PCR. (G) Maximum intensity projections of TUG1 exon (gray) and intron 1 (magenta) or intron 2 (magenta) smRNA FISH in U-2 OS cells transfected with control TMO and with TUG1 TMO1 and TMO2. Nucleus in blue. Scale bar, 5 μm. Towards the right, distribution of nuclear TUG1, cytoplasmic TUG1, intron 1 or 2 retention in TUG1 TMO1 and TMO2 (red) versus control TMO (gray), n=50 cells. (H) Relative cell growth of HeLa and U-2 OS cells transfected with TUG1 TMO1 and TMO2, control TMO or transfection agent only (TA). Representative images of U-2 OS transfected with control TMO or TUG1 TMO1 and TMO2 shown on the left. ***P≤0.001, as evaluated by unpaired t-test versus control TMO; error bars represent SD; minimum three independent measurements.

FIG. 2 Nuclear retention of TUG1 affects gene expression. (A) RNASeq coverage across TUG1 showing read coverage increase across TUG1 intron 1 and intron 2 (highlighted in red boxed) upon TMO1 and TMO2 treatment (triplicate showed). (B) Preventing TUG1 export with TMO1 and TMO2 causes reproducible gene expression changes as shown by comparison of two independent experiments, each done in triplicate. Control TMOs (NTC (SEQ ID NO. 6), SCR_1 (SEQ ID NO. 4) and SCR_2 (SEQ ID NO. 5), serve as controls of cells response to transfection and TMO intake.

FIG. 3 Blocking splicing and nuclear export with TMOs. (A) Ratio of spliced (lower band) and unspliced (upper band) TUG1 RNA assessed by PCR in HeLa transfected with TMO1, TMO2, TMO1 and TMO2, TMO1 labelled with FITC and non-targeting TMO (control). (B) TUG1 exon/intron1 and exon/intron2 RNA imaging in HeLa WT and transfected with non-targeting TMO (control), TMO1 and TMO2, TMO1, TMO2. Exons in red, introns in gray, nuclei in blue.

FIG. 4 TMO-based prevention of TERT (SEQ ID NO. 3) splicing reduces cell growth in vitro. (A) Scheme showing the design of TERT TMO (in red) against the exon11/intron 11 donor splice site. The upper-case red letters refer to thiomorpholino nucleotides and the lower-case letter to a 2′-deoxynucleoside at the 3′ end. (B) Experimental setup to assess the efficiency of TMO-based TERT intron 11 inclusion (RT qPCR and smRNA FISH) and its effect on cell growth. (C) Relative expression of TERT intron 11, spliced TERT (Exon10-Exon11, Exon10-Exon12, Exon11-Exon12), and unspliced TERT (Exon11-Intron11) over GAPDH assessed by RT qPCR. Error bars represent SD, three replicates. (D) Maximum intensity projections of TERT exon (gray) and intron 11 (magenta) smRNA FISH in LN-18 cells transfected with control TMO and TERT TMO. DAPI, blue. Scale bar, 5 μm. On the right, quantification of total TERT (exon signal), unspliced TERT, spliced TERT (ΔI11), and free intron 11 during mitosis of LN-18 cells transfected with control TMO (CTRL) or TERT TMO. n.s.=not significant, ***P≤0.001, as evaluated by unpaired t-test versus control TMO; n=30 cells. (E) Cell growth of LN-18 and HEK293T cells transfected with TERT TMO, control TMO or transfection agent only (TA). Representative images of LN-18 transfected with control TMO or TERT TMO shown on the left. Scale bar, 25 μm. *P≤0.05, **P≤0.01, as evaluated by unpaired t-test versus control TMO; error bars represent SD; minimum two (HEK293T), three (LN-18) independent measurements. (F) TERT TMO-mediated effects on cell viability, cell cycle and genomic damage (γH2AX foci, green) are absent upon co-expression of spliced TERT. Cell viability and cell cycle, 3 independent measurements. γH2AX foci n=128-168 cells from 2 independent experiments.

### DETAILED DESCRIPTION OF THE INVENTION

The present disclosure provides ASOs comprising thiomorpholino nucleotides effective to treat or prevent RNA localization disorders such as cancer by administering thiomorpholino-containing nucleotides (TMOs) that are configured to hybridize with target RNAs, and preferably lncRNAs, in the cell's nucleus after transcription and sterically prevent intron excision and inhibit nuclear export of target RNA to the cell cytoplasm. Accordingly, this disclosure also provides methods of treating one or more RNA localization disorders. As used herein, an “RNA localization disorders” generally include any disease or disorder that may be treated through the administration of a therapeutically effective amount of TMOs that sterically prevents intron excision and prevent nuclear export of a target RNA. In one embodiment, an RNA localization disorder may include cancer, and preferably cancers that may be treated by the subcellular localization of one or more cancer-associated RNAs in the cell cytoplasm.

In another embodiment, the TMOs of this disclosure may be administered in a dose and for a time period to thereby sequester one or more target RNA, and preferably one or more lncRNAs in the cell nucleus. In one preferred embodiment, TMOs of this disclosure may be administered in a dose and for a time period to inhibit decrease intron excision and nuclear export of one or more target RNA in the cell nucleus in a subject to at least 20%, about 30%, about 40%, about 50%, about 60%, about 70%, about 80%, about 90%, about 95%, or more than 95%.

In another embodiment, the TMOs of this disclosure may be administered by systemic administration, such as once weekly or bimonthly, by infusion. In certain embodiments an effective amount of an administered composition may comprise one of several amounts e.g., 2 mg/kg, about 5 mg/kg, about 10 mg/kg, or a dosage in the range of 15 mg/kg to 50 mg/kg, which includes an antisense TMOs as described herein, administered over a period of time sufficient to treat the disease or disorder.

Another embodiment may comprise methods of treating a RNA localization disorder, and preferably cancer, in a subject by administering a composition comprising an antisense TMO of this disclosure of between 20 to 50 nucleotides in length comprising at least 10 consecutive nucleotides complementary to a region of a target RNA, wherein the antisense oligonucleotide specifically hybridizes to the target region including an exon/intron junction or splice site. The hybridization of one or more TMOs to the target or donor RNA may sterically hinder splicing of the target RNA and further inhibit its nuclear localization decreasing the number of excised mRNA that may be translated in the cell, and thereby treating the RNA localization disorder in the subject.

In another embodiment, the anti sense TMO may be complementary to a target region in a target RNA having a splice site. In other embodiments, a plurality of antisense TMOs may be complementary to a plurality of target regions in a target RNA each having a splice site. In more preferred embodiments, the antisense TMO may be complementary to a target region in lncRNAs having a splice site. In other embodiments, a plurality of antisense TMOs may be complementary to a plurality of target regions in lncRNAs each having a splice site.

In one specific embodiment, the antisense TMO may be identified as SEQ ID NO. 1 or 2 and may be complementary to a target region in containing an exon/intron junction splice site for the lncRNAs encoded by for the TUG1 gene. In this specific embodiment, the antisense TMO may be identified as SEQ ID NO. 1 or 2 may be administered to subject in need thereof for the treatment of a RNA localization disorder, such as cancer.

In another embodiment, the antisense TMOs may be chemically linked to one or more moieties or conjugates that enhance the activity, cellular distribution, or cellular uptake of the antisense oligonucleotide, such as an arginine-rich peptide or cell penetrating peptide.

Thus, this disclosure also provides unique antisense oligonucleotides containing at least one TMO, for example as identified in SEQ ID NOs. 1-3, as well as pharmaceutical compositions comprising such TMOs and one or more pharmaceutical excipients that may be directed to the treatment of an RNA localization disorder such as cancer in a subject in need thereof.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “complementary” and “complementarity” refer to polynucleotides (i.e., a sequence of nucleotides) related by base-pairing rules. For example, the sequence “T-G-A (5′-3′),” is complementary to the sequence “T-C-A (5′-3′).” Complementarity may be “partial,” in which only some of the nucleic acids' bases are matched according to base pairing rules. Or there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “antisense oligomer” and “antisense compound” and “antisense oligonucleotide” are used interchangeably and refer to a sequence of cyclic nucleotides, each bearing a base-pairing moiety, linked by internucleotide linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in a preferred embodiment, a thiomorpholino group (see description of morpholino oligomers below). The oligomer may have exact or near sequence complementarity to the target sequence; variations in sequence near the termini of an oligomer are generally preferable to variations in the interior.

In these methods, the ASO, and preferably a TMO, can be designed to block or inhibit natural RNA splice processing and post-processing nuclear localization from the cell's nucleus, and may be said to be “directed to” or “targeted against” a target sequence with which it hybridizes. The target sequence is typically a region including an exon/intron junction or splice site of a pre-processed RNA. A preferred target sequence is any region of a preprocessed RNA that includes an exon/intron junction or splice site. An ASO is more generally said to be “targeted against” a biologically relevant target, such as a protein, virus, or bacteria, when it is targeted against the nucleic acid of the target in the manner described above.

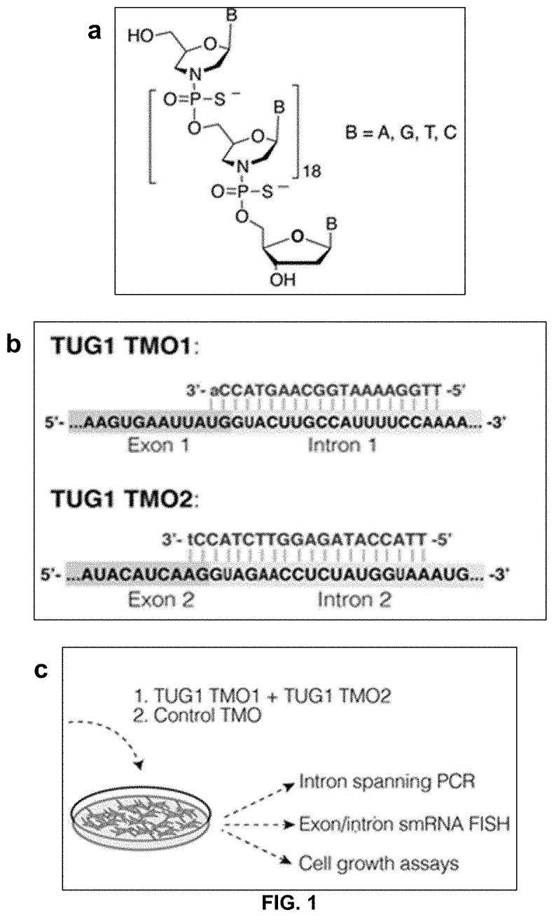

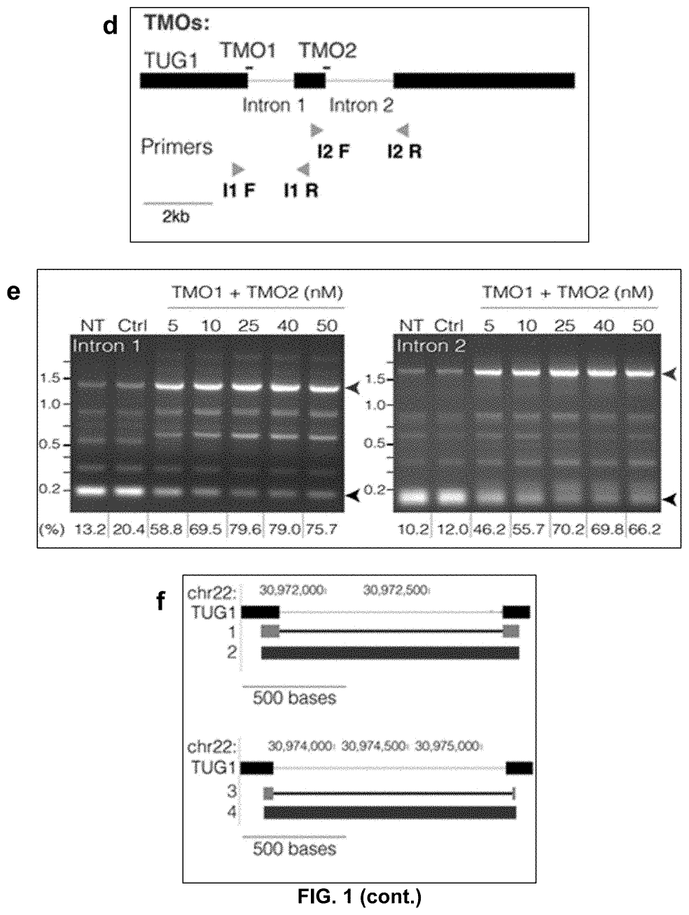

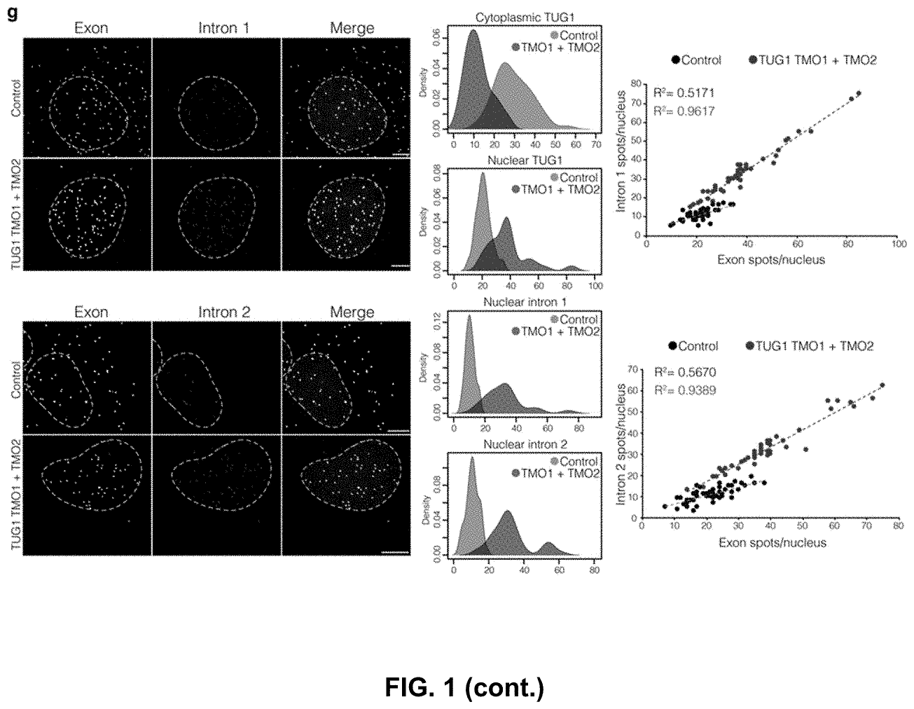

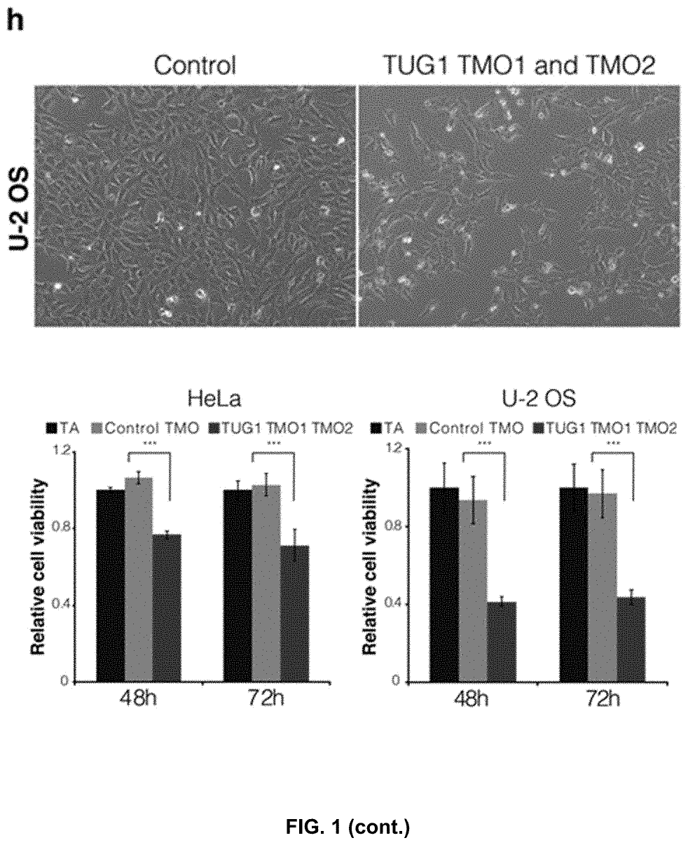

The terms “morpholino oligomer” or “thiomorpholino oligomer” or “thiomorpholino oligonucleotides” or “TMO” refer to an oligonucleotide analog composed of morpholino subunit structures (including thiomorpholino), where (i) the structures are linked together by phosphorothioates-containing linkages, one to three atoms long, preferably two atoms long, that may be uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base-specific hydrogen bonding, to a base in a polynucleotide. See, for example, the structures depicted in FIG. 1. Variations can be made to this linkage as long as they do not interfere with binding or activity. The 5′ oxygen may be substituted with amino or lower alkyl substituted amino. The pendant nitrogen attached to phosphorus may be unsubstituted, monosubstituted, or disubstituted with (optionally substituted) lower alkyl. The purine or pyrimidine base pairing moiety is typically adenine, cytosine, guanine, uracil, thymine or inosine. The synthesis, structures, and binding characteristics of morpholino oligomers are detailed in co-pending PCT Patent Application No's. PCT/US17/51839, filed Sep. 15, 2017, and PCT/US18/51907, filed Sep. 20, 2018, both of which are incorporated herein by reference in their entirety.

An “amino acid subunit” or “amino acid residue” can refer to an alpha-amino acid residue (—CO—CHR—NH—) or a beta- or other amino acid residue (e.g. —CO—(CH₂)ₙCHR—NH—), wherein R is a side chain (which may include hydrogen) and n is 1 to 6, preferably 1 to 4.

The term “naturally occurring amino acid” refers to an amino acid present in proteins found in nature. The term “non-natural amino acids” refers to those amino acids that are not present in proteins found in nature; examples include beta-alanine (beta-Ala), 6-aminohexanoic acid (Ahx) and 6-aminopentanoic acid.

An “exon” refers to a defined section of nucleic acid that encodes a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

A “lncRNA” refers to a 200-100,000 nt long mRNA-like transcripts lacking protein-coding features such as open-reading frames and exert their functional role as RNA transcripts. LncRNAs have altered expression in human cancers.

An “RNA localization disorder” refers to a disease or condition that may be treated by the administration of chemically modified ASOs, and preferably one or more TMOs, to block RNA splicing and sequester clinically significant target RNA, and preferably lncRNAs in the cell nucleus by inhibiting nuclear transport. Various cancers are examples of RNA localization disorders.

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, and isolated RNA of any sequence, recombinant polynucleotides, primers, probes, plasmids, and vectors. Included within the definition are nucleic acid polymers that have been modified, whether naturally or by intervention.

The therapeutic ASO compositions of the invention may be also be an effective treatment for numerous specific cancer or other disease conditions including without limitation, bladder cancer, lung cancer, head and neck cancer, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, memngioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, and leukemia.

Optionally, the therapeutic methods and ASO compositions, such as TMOs, of the present invention may be combined with other anti-cancer therapies. Examples of anti-cancer therapies include traditional cancer treatments such as surgery and chemotherapy, as well as other new treatments. Such other anti-cancer therapies will be expected to act in an additive or synergistic manner with the ASO therapy. This may result in better control of the cancer as well as reducing the need for high dosages of chemotherapeutic or radiation therapies. For example, a wide array of conventional compounds, have been shown to have anti-cancer activities. These compounds have been used as pharmaceutical agents in chemotherapy to shrink solid tumors, prevent metastases and further growth, or decrease the number of malignant cells in leukemic or bone marrow malignancies.

An “effective amount” or “therapeutically effective amount” refers to an amount of therapeutic compound, such as an antisense oligonucleotide, administered to a human subject, either as a single dose or as part of a series of doses, which is effective to produce a desired therapeutic effect. For an antisense oligonucleotide, this effect is typically brought about by inhibiting translation or natural splice-processing of a selected target sequence. An effective amount may be variable such as 5 mg/kg of a composition comprising a TMO for a period of time to treat the subject. In one embodiment, an effective amount might be 5 mg/kg of a composition comprising an antisense oligonucleotide to maintain or increase sequestration of the target RNA in the cell's nucleus. In another embodiment, an effective amount may be 5 mg/kg of a composition including an antisense oligonucleotide to maintain or increase sequestration of the target RNA in the cell's nucleus, relative to a healthy peer. In another embodiment, an effective amount may be 5 mg/kg, administered for at least 24 weeks, at least 36 weeks, or at least 48 weeks, to thereby maintain or increase sequestration of the target RNA in the cell's nucleus in a subject to at least 20%, to about 30%, about 40%, about 50%, about 60%, about 70%, about 80%, about 90%, or to about 95% of normal.

As used herein, “sufficient length” refers to an antisense oligonucleotide that is complementary to at least 8, more typically 8-30, contiguous nucleobases in a target RNA, such as a lncRNA. In some embodiments, an antisense oligonucleotide of sufficient length includes at least 8, 9, 10, 11, 12, 13, 14, or 15 contiguous nucleobases in a target RNA, for example the lncRNAs TUG1. In other embodiments, an antisense of sufficient length includes at least 16, 17, 18, 19, 20, 21, 22, 23, 24, or 25 contiguous nucleobases in a target RNA, for example the lncRNAs TUG1. An antisense oligonucleotide of sufficient length has at least a minimal number of nucleotides to be capable of specifically hybridizing to any one or more of exons and/or intron site, for example one or more exon or intron portions of a lncRNAs like TUG1. Preferably, the antisense oligonucleotide of the invention has a minimal number of nucleotides to be capable of specifically hybridizing to any one or more of intron or exon portions of a target RNA and alter its subcellular localization, and preferably by blocking nuclear export out of the cell's nucleus. Preferably an oligonucleotide of sufficient length is from about 8 to about 50 nucleotides in length, including oligonucleotides of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. Thus, an oligonucleotide of sufficient length may be from 10 to about 30 nucleotides in length, or from 15 to about 25 nucleotides in length, or from 20 to 30, or 20 to 50, nucleotides in length, or from 25 to 28 nucleotides in length.

Also included are vector delivery systems that are capable of expressing the TMO of the present invention, such as vectors that express a polynucleotide sequence, and variants thereof, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

“Treatment” of an individual (e.g., a mammal, such as a human) or a cell is any type of intervention used in an attempt to alter the natural course of the individual or cell, for example by sequestering target splicing RNAs in the cell's nucleus or, in other words preventing nuclear export of target splicing RNAs from the cell's nucleus. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with a target splicing RNA that may be sequestered in the cell's nucleuses, as in certain forms of cancer, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

As used herein, the terms “introducing” means brining into contact with one or more components of a cell in vivo or in vitro. In a preferred embodiment, introducing may be through administering a therapeutically effective amount of an ASO of the invention to a subject in need thereof.

In one embodiment, treatment with antisense TMOs of the invention may hybridize with and sterically hinder RNA splicing events in the nucleus thereby preventing nuclear export of the hybridized RNA and TMOs. In one preferred embodiment, treatment with antisense TMOs of the invention may hybridize with and sterically hinder splicing of a target RNA, and preferably a lncRNAs associated with a RNA localization disorder, such as cancer cell growth caused by mutations in, and/or upregulation of clinically relevant RNAs such as TUG1, TERT, and KRAS, among others.

As used herein, a “subject” includes any animal, and preferably a human, that exhibits a symptom, or is at risk for exhibiting a symptom, which can be treated with an antisense compound of the invention, such as a subject that has or is at risk for having an RNA localization disorder, such as cancer.

The antisense TMOs of this disclosure may include oligonucleotide moieties conjugated to a CPP, preferably an arginine-rich peptide transport moiety effective to enhance transport of the compound into cells. The transport moiety is preferably attached to a terminus of the oligomer. The peptides have the capability of inducing cell penetration within 30%, 40%, 50%, 60%, 70%, 80%, 90%, or 100% of cells of a given cell culture population, including all integers in between, and allow macromolecular translocation within multiple tissues in vivo upon systemic administration. In one embodiment, the cell-penetrating peptide may be an arginine-rich peptide transporter. In another embodiment, the cell-penetrating peptide may be Penetratin or the TAT peptide. These peptides are well known in the art and are disclosed, for example, in US Patent Publication No. 2010/0016215, incorporated herein by reference in its entirety. A particularly preferred approach to conjugation of peptides to antisense oligonucleotides can be found in PCT publication No. WO2012/150960, which is incorporated herein by reference in its entirety. A preferred embodiment of a peptide conjugated oligonucleotides of this disclosure utilizes glycine as the linker between the CPP and the antisense oligonucleotide. For example, a preferred peptide conjugated PMO consists of R₆-G-TMO. These transport moieties have been shown to greatly enhance cell entry of attached oligomers, relative to uptake of the oligomer in the absence of the attached transport moiety. Uptake is preferably enhanced at least ten-fold, and more preferably twenty-fold, relative to the unconjugated compound.

This disclosure also provides formulations or compositions suitable for the therapeutic delivery of antisense oligomers to a subject. These compositions may be pharmaceutically acceptable compositions that comprise a therapeutically-effective amount of one or more of the oligomers described herein, formulated together with one or more pharmaceutically acceptable carriers (additives) and/or diluents. While it is possible for an oligomer of this disclosure to be administered alone, it is preferable to administer the compound as a pharmaceutical formulation (composition).

The TMOs of this disclosure may be administered alone or in combination with another therapeutic. The additional therapeutic may be administered prior to, concurrent with, or subsequent to the administration of the compositions of the present invention. For example, the compositions may be administered in combination with anti-cancer chemotherapeutic agents, or radiation therapies for example.

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including the isolated oligomers of the present invention.

As detailed below, the pharmaceutical compositions of this disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

Examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligomers of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of mesoporus silica, or polybutylcyanoacrylate, which can deliver drugs across the blood brain barrier and can alter neuronal uptake mechanisms (Prog Neuropsychopharmacol Biol Psychiatry, 23, 941-949, 1999).

These compositions may also comprise liposomes or lipoplexes, including surface-modified liposomes/lipoplexes containing saccharides and/or poly(ethylene glycol) lipids (PEG-modified, branched and unbranched or combinations thereof, or long-circulating liposomes or stealth liposomes). Oligomers of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug. Such liposomes/lipoplexes have been shown to accumulate selectively in tumors. The long-circulating liposomes/lipoplexes may enhance the pharmacokinetics and pharmacodynamics of DNA and RNA, particularly compared to conventional cationic liposomes. Long-circulating liposomes are also likely to protect drugs from nuclease degradation to a greater extent compared to cationic liposomes, based on their ability to avoid accumulation in metabolically aggressive MPS tissues such as the liver and spleen.

This disclosure includes oligomer compositions prepared for delivery as described in U.S. Pat. Nos. 6,692,911, 7,163,695 and 7,070,807. Thus, this disclosure provides an oligomer of this disclosure in a composition comprising copolymers of lysine and histidine (HK) (as described in U.S. Pat. Nos. 7,163,695; 7,070,807; and 6,692,911) either alone or in combination with PEG (e.g., branched or unbranched PEG or a mixture of both), in combination with PEG and a targeting moiety or any of the foregoing in combination with a crosslinking agent. This disclosure also provides antisense oligomers in compositions comprising gluconic-acid-modified polyhistidine or gluconylated-polyhistidine/transferrin-polylysine. Amino acids with properties similar to His and Lys may be substituted within the composition.

The oligomers described herein may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the subject oligomers include the conventional nontoxic salts or quaternary ammonium salts of the compounds, e.g., from non-toxic organic or inorganic acids. For example, conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The oligomers of this disclosure may contain one or more acidic functional groups and, thus, are capable of forming pharmaceutically-acceptable salts with pharmaceutically-acceptable bases. The term “pharmaceutically-acceptable salts” in these instances refer to the relatively non-toxic, inorganic and organic base addition salts of the TMO compounds of this disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in these compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Useful formulations of this disclosure include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, and the mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

A formulation of this disclosure may comprise an excipient selected from cyclodextrins, celluloses, liposomes, micelle forming agents, e.g., bile acids, and polymeric carriers, e.g., polyesters and polyanhydrides; and an oligomer of the present invention, that may render orally bioavailable an oligomer of this disclosure.

Methods of preparing these formulations or compositions include the step of bringing into association an oligomer of this disclosure with the carrier and, optionally, one or more accessory ingredients. In general, the formulations are prepared by uniformly and intimately bringing into association a compound of this disclosure with liquid carriers, or finely divided solid carriers, or both, and then, if necessary, shaping the product.

The formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of this disclosure as an active ingredient. An oligomer of this disclosure may also be administered as a bolus, electuary or paste.

In these solid dosage forms for oral administration (capsules, tablets, pills, dragees, powders, granules, trouches and the like), the active TMO therapeutic ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active oligomers may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligomer of the present invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of this disclosure to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more oligomers of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by using coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by using a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsulated matrices of the subject oligomers in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also be prepared by entrapping the drug in liposomes or microemulsions that are compatible with body tissues.

When the oligomers of this disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations of this disclosure may be given orally, parenterally, systemically, topically, rectally, or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the oligomers of the present invention, which may be used in a suitable hydrated form, and/or the pharmaceutical compositions of the present invention, may be formulated into pharmaceutically-acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of this disclosure employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and gradually increase the dosage until the desired effect is achieved. In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular, intramuscular and subcutaneous doses of the compounds of this invention for a patient, when used for the indicated effects, will range from about 0.0001 to about 100 mg per kilogram of body weight per day.

Preferred doses of the thiomorpholino oligomers of this disclosure are administered generally from about 5-100 mg/kg. In some cases, doses of greater than 100 mg/kg may be necessary. For i.v. administration, preferred doses are from about 0.1 mg to 100 mg/kg. In some embodiments, the thiomorpholino oligomers are administered at doses of about 2 mg/kg, to about 100 mg/kg, including all integers in between.

If desired, the effective daily dose of the active compound may be administered as two, three, four, five, six or more sub-doses administered separately at appropriate intervals throughout the day, optionally, in unit dosage forms. In certain situations, dosing is one administration per day. The dosing frequency is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired nuclear localization of the target RNA.

In some embodiments, the oligomers of this disclosure are administered, generally at regular intervals (e.g., daily, weekly, biweekly, monthly, bimonthly). The oligomers may be administered at regular intervals, e.g., daily; once every two days; once every three days; once every 3 to 7 days; once every 3 to 10 days; once every 7 to 10 days; once every week; once every two weeks; once monthly. For example, the oligomers may be administered once weekly by intravenous infusion. The oligomers may be administered intermittently over a longer period, e.g., for several weeks, months or years. For example, the oligomers may be administered once every one, two, three, four, five, six, seven, eight, nine, ten, eleven or twelve months. In addition, the oligomers may be administered once every one, two, three, four or five years. Administration may be followed by, or concurrent with, administration of an antibiotic, steroid or other therapeutic agent. The treatment regimen may be adjusted (dose, frequency, route, etc.) as indicated, based on the results of immunoassays, other biochemical tests and physiological examination of the subject under treatment.

Nucleic acid molecules can be administered to cells by a variety of methods known to those familiar to the art, including, but not restricted to, encapsulation in liposomes or lipoplexes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Among other benefits, microemulsification provides enhanced bioavailability by preferentially directing absorption to the lymphatic system instead of the circulatory system, which thereby bypasses the liver, and prevents destruction of the compounds in the hepatobiliary circulation.

While all suitable amphiphilic carriers are contemplated, the presently preferred carriers are generally those that have Generally-Recognized-as-Safe (GRAS) status, and that can both solubilize the compound of this disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethylene-glycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or monounsaturated fatty acids (SPAN-series) or corresponding ethoxylated analogs (TWEEN-series).

The delivery may occur by use of liposomes, lipoplexes, nanocapsules, microparticles, microspheres, lipid particles, vesicles, and the like, for the introduction of the compositions of this disclosure into suitable host cells. In particular, the compositions of this disclosure may be formulated for delivery either encapsulated in a lipid particle, a liposome, a lipoplex, a vesicle, a nanosphere, a nanoparticle, or the like. The formulation and use of such delivery vehicles can be carried out using known and conventional techniques.

Hydrophilic polymers which may be suitable for use in this disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

A formulation of this disclosure may comprise a biocompatible polymer selected from the group consisting of polyamides, polycarbonates, polyalkylenes, polymers of acrylic and methacrylic esters, polyvinyl polymers, polyglycolides, polysiloxanes, polyurethanes and copolymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letters alpha, beta, or gamma, respectively. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds. The complexation takes place by Van der Waals interactions and by hydrogen bond formation. The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 micrometers in diameter; large unilamellar vesicles (LUVS) are typically larger than 0.05 micrometers Oligolamellar large vesicles and multilamellar vesicles have multiple, usually concentric, membrane layers and are typically larger than 0.1 micrometers. Liposomes with several nonconcentric membranes, i.e., several smaller vesicles contained within a larger vesicle, are termed multivesicular vesicles. Thus, formulations comprising liposomes containing a thiomorpholino oligomer of this disclosure, where the liposome membrane is formulated to provide a liposome with increased carrying capacity. Alternatively, or additionally, the compound of this disclosure may be contained within, or adsorbed onto, the liposome bilayer of the liposome. An oligomer of this disclosure may be aggregated with a lipid surfactant and carried within the liposome's internal space; in these cases, the liposome membrane is formulated to resist the disruptive effects of the active agent-surfactant aggregate. The lipid bilayer of these liposomes may contain lipids derivatized with a saccharide, including a disaccharide such as lactose, a polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of this disclosure are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present invention.

Liposomes according to this disclosure may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers BV, Amsterdam, 1993. For example, liposomes of this disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one embodiment of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT™ and Lipofectamine™ may be utilized to introduce polynucleotides or proteins into cells.

The release characteristics of a formulation of this disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer).

Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween™ and Pluronic™. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

In addition to the methods provided herein, the oligomers for use according to the invention may be formulated for administration in any convenient way for use in human or veterinary medicine, by analogy with other pharmaceuticals. The antisense oligomers and their corresponding formulations may be administered alone or in combination with other therapeutic strategies in the treatment of RNA localization disorders, such as cancer.

### EXAMPLES

### Example 1: Design of TMOs Configured to Promote Nuclear Retention of Splicing RNAs

The present inventors have developed novel systems and compositions for the use of ASO configured to allow nuclear retention of splicing RNAs. To achieve this, chemically modified ASOs were used to prevent splicing. Specifically, chemically modified ASOs called thiomorpholino oligonucleotides (TMOs), containing morpholino rings joined through thiophosphate internucleotide linkages (FIG. 1a) were synthesized. TMOs of 20 nucleotides in length were designed complementary to the two donor splice sites of TUG1 RNA (TMO1 and TMO2, FIG. 1b). TMOs were designed to hybridize against 2 nucleotides within the exon and 18 nucleotides within the intron. Sterically inhibiting the donor splice sites prevented splicing, caused retention of the corresponding intron and nuclear retention of the unspliced RNA. Specifically, transfecting both TMOs into human cell lines prevents excision of both introns from TUG1 RNA and leads to nuclear retention of virtually all unspliced TUG1 RNA (FIG. 1e-g).

### Example 2: Nuclear Retention of Splicing RNAs Alters Global Gene Expression

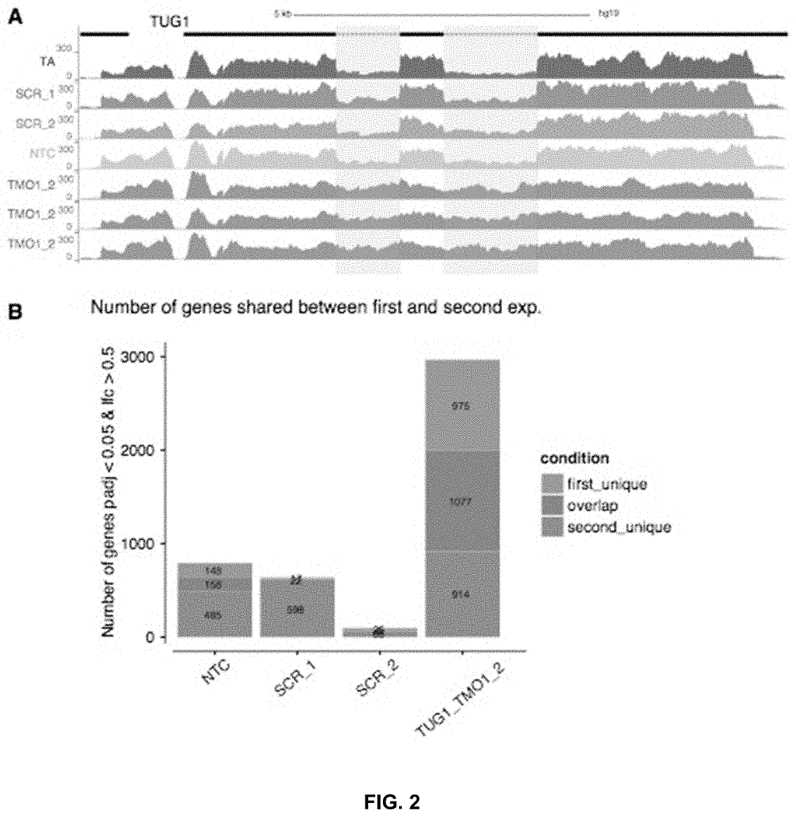

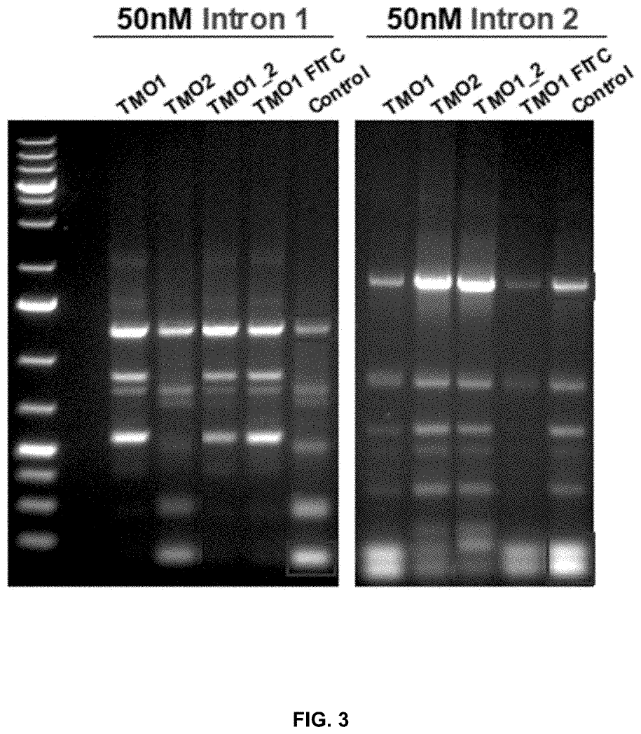

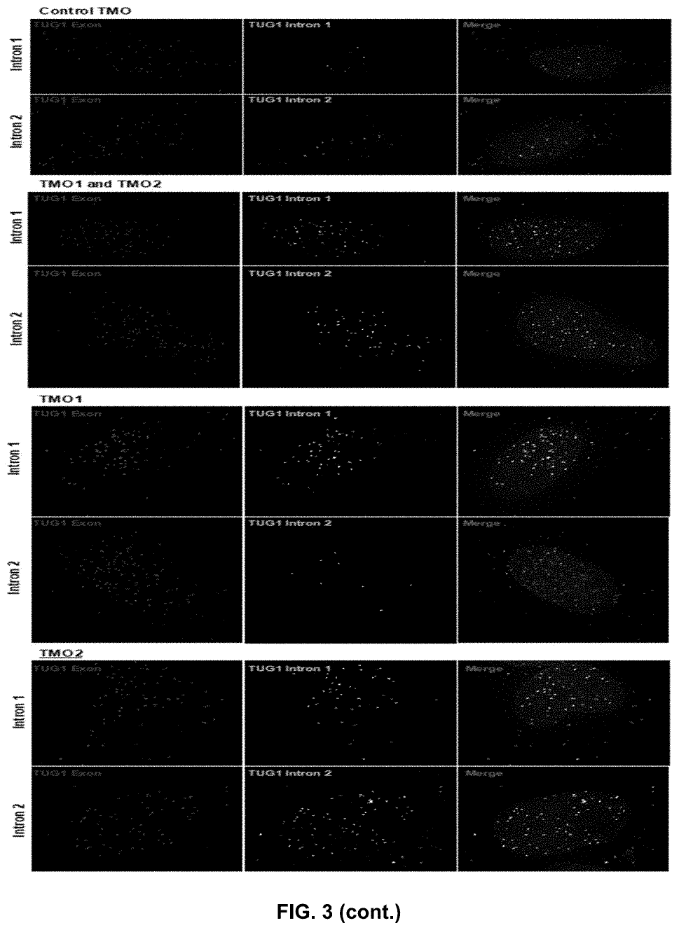

As shown in FIGS. 1h, and 2, altering subcellular localization of TUG1 RNA with TMOs (SEQ ID NO.'s 1-2) reduced cell viability and affected gene expression. For comparative purposes, 3 non-targeting TMOs were designed to account for background gene changes in the cell due to transfection and oligonucleotide intake, thereby facilitating finding genes affected by changes of the RNA location. As shown in FIG. 3, to achieve nuclear enrichment, both TMOs or individual TMOs complementary to either the first or the second TUG1 donor splice site were applied. Individual TMOs prevented excision of just first or the second intron, and in both instances caused nuclear retention of the unspliced RNA.

### Example 3: Retention of TERT mRNA in the Nucleus

Telomerase reverse transcriptase (TERT) mRNA, whose encoded protein extends the ends of chromosomes, is mostly retained in the nucleus in stem cells and cancer cells. Nuclear TERT is partially unspliced, retaining certain introns, intron 11 and intron 14. TERT is silenced in adult somatic tissues and reactivated in the majority of cancers allowing indefinite cell proliferation and cancer progression. Thus, using TMOs to entirely retain TERT mRNA in the nucleus all novel cancer therapies.

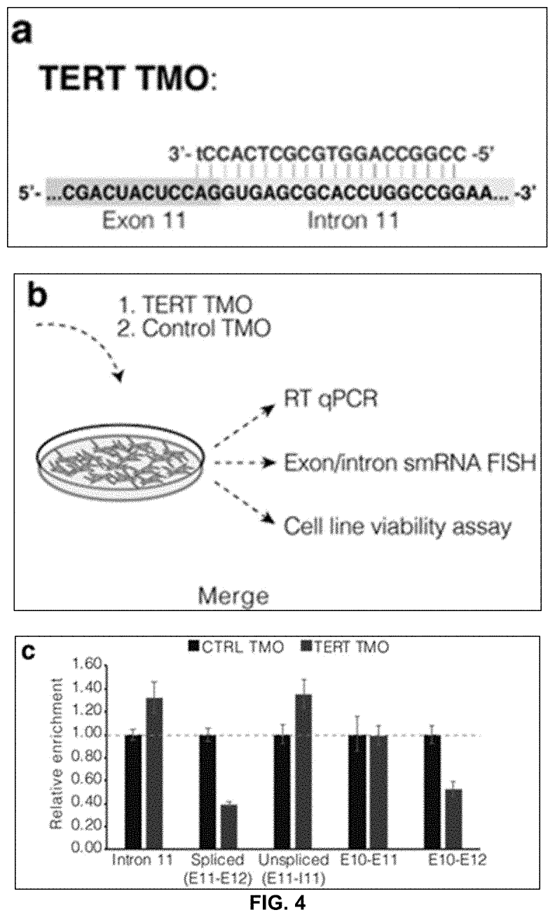

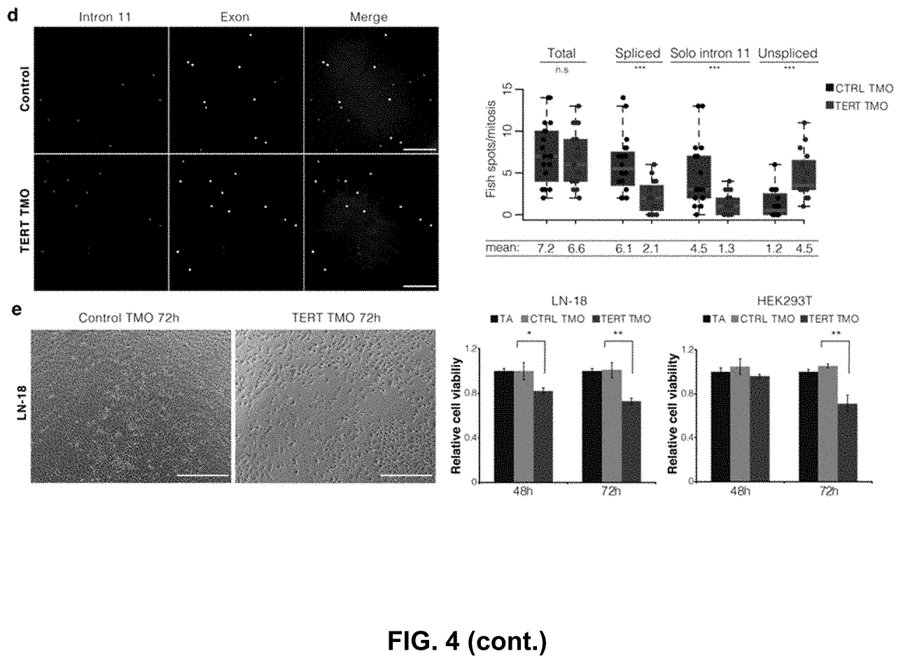

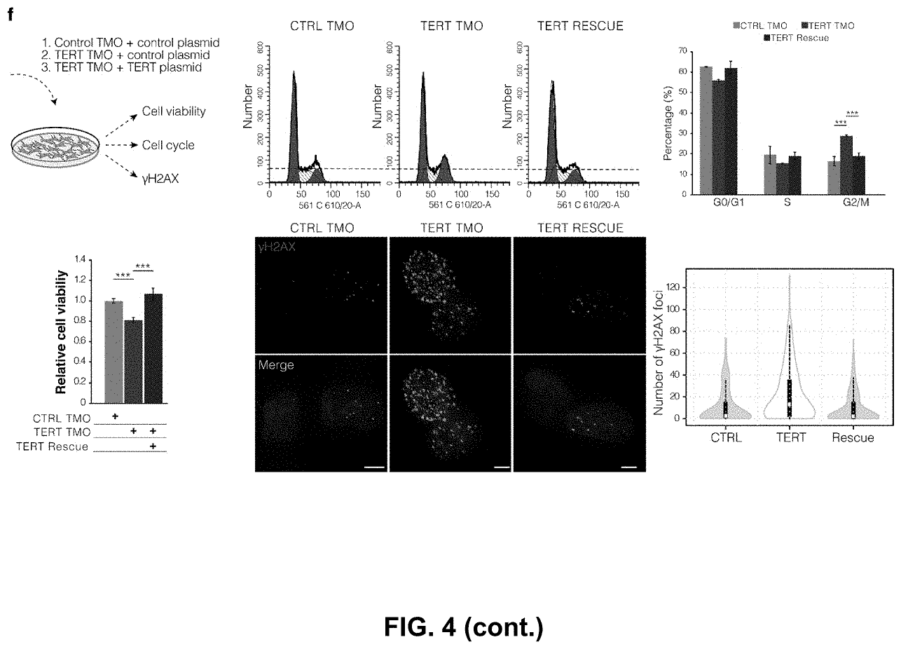

As shown in FIG. 4a-b, TMOs were used to prevent splicing. Specifically, TMOs of 20 nucleotides in length were designed complementary to the exon 11/intron 11 donor splice site of TERT pre-mRNA (SEQ ID NO. 3). TMOs were designed to hybridize against 2 nucleotides within the exon and 18 nucleotides within the intron. Inhibiting the splicing of intron 11 reduced the quantity of spliced TERT mRNA (FIG. 4c,d). Reducing the quantity of spliced TERT with TMOs reduced cell viability compared to control TMO. The specificity of TERT TMO effect was validated by co-expressing a plasmid with spliced TERT (FIG. 4f).

### Example 4: Design and Use of TMOs as Therapeutic Agents for the Treatment of Disease

These findings demonstrate that chemically modified ASOs, such as TMOs can be used to alter the subcellular localization of an RNA by preventing splicing and causing intron retention. As noted above, this approach is broadly applicable beyond the exemplary TUG1 and TERT. Broader applications to other non-coding and protein-coding transcripts are anticipated. Using TMOs to entirely quarantine clinically relevant RNAs in the nucleus by force retention, such as various oncogenes, would be a beneficial tool and open new avenues for disease therapies. For instance, TERT mRNA, whose encoded protein extends the ends of chromosomes, is mostly retained in the nucleus in stem cells and cancer cells. TERT is silenced in adult somatic tissues and reactivated in the majority of cancers allowing indefinite cell proliferation and cancer progression. Using TMOs to entirely retain TERT mRNA in the nucleus would open new avenues for cancer therapies.

### REFERENCES

The following are hereby incorporated by reference:
1. Djebali, S. et al. Landscape of transcription in human cells. Nature 489, 101-108 (2012).
2. Hangauer, M. J., Vaughn, I. W. & McManus, M. T. Pervasive Transcription of the Human Genome Produces Thousands of Previously Unidentified Long Intergenic Noncoding RNAs. PLoS Genet. 9, (2013).
3. Hindorff, L. A. et al. Potential etiologic and functional implications of genome-wide association loci for human diseases and traits. Proc. Natl. Acad. Sci. U.S.A 106, 9362-9367 (2009).
4. Jin, G. et al. Human polymorphisms at long non-coding RNAs (lncRNAs) and association with prostate cancer risk. Carcinogenesis 32, 1655-1659 (2011).
5. Du, Z. et al. Integrative genomic analyses reveal clinically relevant long noncoding RNAs in human cancer.

Nat. Struct. Mol. Biol. 20, 908-913 (2013).
6. White, N. M. et al. Transcriptome sequencing reveals altered long intergenic non-coding RNAs in lung cancer. Genome Biol. 15, 429 (2014).
7. Sauvageau, M. et al. Multiple knockout mouse models reveal lincRNAs are required for life and brain development. Elife 2, 1-24 (2013).
8. Young, T. L., Matsuda, T. & Cepko, C. L. The Noncoding RNA Taurine Upregulated Gene 1 Is Required for Differentiation of the Murine Retina 77 Avenue Louis Pasteur. Curr. Biol. 15, 501-512 (2005).
9. Katsushima, K. et al. Targeting the Notch-regulated non-coding RNA TUG1 for glioma treatment. Nat. Commun. 7, 1-14 (2016).
10. Jianyin, L. et al. Long noncoding RNA Tug1 regulates mitochondrial bioenergetics in diabetic nephropathy. J. Clin. Invest. 126, 4205-4218 (2016).
11. He, Q. et al. Long noncoding RNA TUG1 facilitates osteogenic differentiation of periodontal ligament stem cells via interacting with Lin28A. Cell Death Dis. 9, (2018).
12. Lewandowski, J. P. et al. The Tug1 Locus is Essential for Male Fertility. bioRxiv 1-44 (2019).
13. Dominskil, Z. & Ryszard, K. Restoration of correct splicing in thalassemic pre-mRNA by antisense oligonucleotides. PNAS 90, 8673-8677 (1993).
14. Scaffidi, P. & Misteli, T. Reversal of the cellular phenotype in the premature aging disease Hutchinson-Gilford progeria syndrome. Nat. Med. 11, 440-445 (2005).
15. McClorey, G., Moulton, H. M., Iversen, P. L., Fletcher, S. & Wilton, S. D.

Antisense oligonucleotide-induced exon skipping restores dystrophin expression in vitro in a canine model of DMD. Gene Ther. 13, 1373-1381 (2006).
16. Pros, E. et al. Antisense therapeutics for neurofibromatosis type 1 caused by deep intronic mutations. Hum. Mutat. 30, 454-462 (2009).
17. Rowland, T. J., Dumbović, G., Hass, E. P., Rinn, J. L. & Cech, T. R. Single-cell imaging reveals unexpected heterogeneity of TERT expression across cancer cell lines. PNAS 116, 18488-18497 (2019).
18. Ryan, M. B., Corcoran, R. B. Therapeutic strategies to target RAS-mutant cancers. Nat Rev Clin Oncol 15, 709-720 (2018) doi:10.1038/s41571-018-0105-0

SEQUENCE LISTING

SEQ ID NO. 1
DNA
TMO1-antisense TMO
Artificial
A*C*C*A*T*G*A*A*C*G*G*T*A*A*A*G*G*T*T
SEQ ID NO. 2
DNA
TMO1-antisense TMO
Artificial
T*C*C*A*T*C*T*T*G*G*A*G*A*T*A*C*C*A*T*T
SEQ ID NO. 3
DNA
TERT-antisense TMO
Artificial
T*C*C*A*C*T*C*G*C*G*R*G*G*A*C*C*G*G*C*C
SEQ ID NO. 4
DNA
SCR_1-control
Artificial
A*C*A*C*G*G*A*T*A*T*C*G*G*T*A*A*G*A*A*T
SEQ ID NO. 5
DNA
SCR_2-control
Artificial
G*A*C*T*T*A*G*A*C*A*A*T*T*A*A*C*G*A*A*G
SEQ ID NO. 6
DNA
NTC-control
Artificial
G*A*G*T*C*A*T*T*C*G*A*C*T*T*C*T*G*A*C*T
*indicates thiomorpholino internucleotide linkage

1-21. (canceled)

22. A method of altering subcellular localization of target RNA comprising the steps of:
introducing to a cell a composition comprising at least one thiomorpholino oligonucleotides (TMO), comprising at least one thiomorpholino nucleotide, and wherein the TMO is configured to be complementary to a target region on a target RNA wherein the TMO specifically hybridizes to the target region thereby sterically inhibiting RNA splicing causing nuclear retention of the unspliced RNA.

23. The method of claim 22, wherein the target region on the target RNA is an exon/intron junction.

24. The method of claim 22, wherein the target region on the target RNA is a region that is not an exon/intron junction.

25. The method of claim 22, wherein the TMO comprises at least 2 consecutive nucleotides complementary to an exon sequence of the target region, and at least 18 consecutive nucleotides complementary to an intron sequence of the target region.

26. The method of claim 22, wherein the TMO comprises at least 2 consecutive nucleotides complementary to an exon sequence of the target region, and at least 18 consecutive nucleotides complementary to an intron sequence of the target region.

27. The method of claim 22, wherein the TMO comprises morpholino subunits linked by thiophosphate-containing internucleotide linkages joining a morpholino nitrogen of one residue to a 5′ exocyclic carbon of an adjacent residue.

28. The method of claim 22, wherein the TMO comprises at least 20 morpholino subunits linked by thiomorpholino-containing internucleotide linkages joining a morpholino nitrogen of one residue to a 5′ exocyclic carbon of an adjacent residue.

29. The method of claim 22, wherein the TMO comprises thiomorpholino subunits and at least one of phosphorodiamidate morpholino and thiophosphate internucleotide linkages.

30. The method of claim 22, wherein the target RNA comprises a lncRNA.

31. The method of claim 30, wherein the target lncRNA comprises a TUG1 lncRNA.

32. The method of claim 22, wherein the target RNA comprises one or more lncRNAs associated with cancer.

33. The method of claim 22, wherein the target RNA comprises a pre-processed RNA associated with cancer.

34. The method of claim 33, wherein the pre-processed RNA associated with cancer is selected from the group consisting of: a pre-processed TERT mRNA, and a pre-processed KRAS mRNA.

35. The method of claim 22, wherein the TMO is selected from the group consisting of: as SEQ ID NO.'s 1-3, or a combination of the same.

36-59. (canceled)

60. A method of altering subcellular localization of a target RNA in a cancer cell comprising:
introducing a thiomorpholino oligonucleotide (TMO) having at least one thiomorpholino nucleotide to a cancer cell, and wherein the TMO is configured to be complementary to a target region on a TERT RNA wherein the TMO specifically hybridizes to said target region thereby sterically inhibiting RNA splicing causing nuclear retention of the unspliced TERT RNA.

61. The method of claim 60, wherein the TMO comprises the TMO according to SEQ ID NO. 3 and wherein said target region comprises the exon11/intron11 donor splice site.

62. A pharmaceutical composition comprising the TMO of claim 61, and at least one pharmaceutically acceptable additive.

63. A method of altering subcellular localization of a target lncRNA in a cancer cell comprising:
introducing an thiomorpholino oligonucleotide (TMO) having at least one thiomorpholino nucleotide, and wherein the TMO is configured to be complementary to a target region on a TUG1 lncRNA wherein the TMO specifically hybridizes to said target region thereby sterically inhibiting RNA splicing causing nuclear retention of the unspliced TUG1 lncRNA.

64. The method of claim 63, wherein the TMO comprises an TMO selected from the group consisting of: SEQ ID NO's. 1, and 2 or a combination of the same.

65. A pharmaceutical composition comprising the TMO of claim 64, and at least one pharmaceutically acceptable additive.
